(19) United States
(12) Patent Application Publication
(10) Pub. No.: US 2022/0184121 A1
(43) Pub. Date: Jun. 16, 2022

(54) AUGMENTATION OF T-CELL ACTIVATION BY OSCILLATORY FORCES AND ENGINEERED ANTIGEN-PRESENTING CELLS

(71) Applicant: THE REGENTS OF THE UNIVERSITY OF CALIFORNIA, Oakland, CA (US)

(72) Inventors: Manish BUTTE, Los Angeles, CA (US); Fatemeh MAJEDI, Los Angeles, CA (US); Mohammad Mahdi HASANI-SADRABADI, Los Angeles, CA (US)

(73) Assignee: THE REGENTS OF THE UNIVERSITY OF CALIFORNIA, Oakland, CA (US)

(21) Appl. No.: 17/440,116

(22) PCT Filed: Mar. 18, 2020
(86) PCT No.: PCT/US2020/023407

§ 371 Date: Sep. 16, 2021

Related U.S. Application Data
(60) Provisional application No. 62/820,067, filed on Mar. 18, 2019.

(51) Int. Cl. A61K 35/17; C12N 5/0783

(52) U.S. Cl. CPC A61K 35/17; C12N 2501/515; C12N 2501/51; C12N 5/0638

(57) ABSTRACT

Aspects of the present disclosure provide methods and compositions for immune cell activation. Disclosed are antibody-coated microparticles and methods for use. In some cases, immune cell activation methods comprising mechanical stimulation are disclosed. Embodiments are directed to activation of cytotoxic T cells. Additional aspects include generation and activation of regulatory T cells.

### CROSS-REFERENCE TO RELATED APPLICATIONS

This application claims priority to U.S. Provisional Patent Application No. 62/820,067, filed Mar. 18, 2019, which application is hereby incorporated by reference in its entirety.

### BACKGROUND

This invention was made with government support under R01GM110482 awarded by the National Institutes of Health. The government has certain rights in the invention.

### I. FIELD OF THE INVENTION

Aspects of this invention relate to the fields of molecular biology and immunology.

### II. BACKGROUND

T lymphocytes circulate throughout the body and coordinate the immune response against pathogens by recognizing their proteome as foreign. Activation of T cells begins by T-cell receptors (TCRs) engaging with antigenic peptides proffered by the major histocompatibility complex (p-MHC) of antigen presenting cells (APCs). Ex vivo cultivation of T cells is important for manufacturing cellular therapies, such as CAR-T cells. Therefore, the optimization of approaches for polyclonal T-cell cultivation is clinically important. In the activation of T cells for biomedical engineering applications, such T cells are commonly cultured with beads coated with stimulatory antibodies, or artificial antigen presenting cells (aAPCs). The key to T-cell activation is offering simulation to the TCR, either in the form of pMHC or by using antibodies that trigger the CD3 chains of the TCR complex. Naive T cells also require costimulation of the CD28 receptor for complete activation, and so antibodies that cross-link and activate CD28 are almost always included in the formulation of aAPCs. The amount of antibodies provided by the aAPC is proportionate to their cost, and so many approaches have attempted to identify and minimize the amount of signal needed.

There remains a need for compositions and methods for ex vivo cultivation and stimulation of T cells with high potency and limited cost.

### SUMMARY

Aspects of the present disclosure provide methods and compositions for effective ex vivo and in vitro activation of T cells. Disclosed are antibody-coated microparticles capable of activating T cells, and methods of using antibody-coated microparticles for T cell activation. Certain embodiments include activation of conventional T cells (e.g., cytotoxic T cells), which may be useful in therapeutic methods such as cancer treatment. Additional embodiments include activation of regulatory T cells, which may be useful in therapeutic methods such as treatment of autoimmune disorders. Microparticles of the present disclosure may be designed and used for activation of various types of T cells by modification of properties such as size, antibody density, and composition. In particular embodiments, the present disclosure includes providing mechanical stimulation, such as oscillatory stimulation, to T cells and antibody-coated microparticles, thereby maximizing the efficacy of T cell activation.

Embodiments of the disclosure include methods for activating an immune cell, methods for activating a T cell, methods for activating a conventional T cell, methods for activating a regulatory T cell, methods for generating an induced regulatory T cell, methods for expanding immune cells, methods for expanding T cells, methods for generating therapeutic T cells, methods for expanding chimeric antigen receptor T cells, methods for treating cancer, methods for treating a viral infection, methods for treating an autoimmune disorder, methods for treating an inflammatory disorder, methods for generating a microparticle, methods for generating an antibody-coated microparticle, methods for generating an artificial antigen presenting cell, and compositions comprising antibody-coated microparticles. Any one or more of these may be excluded from embodiments of the present disclosure.

Methods of the present disclosure can include at least 1, 2, 3, 4, 5, 6, 7, 8, or more of the following steps: generating a mixture comprising an immune cell and an antibody-coated microparticle, generating a mixture comprising a T cell and an antibody-coated microparticle, generating a mixture comprising a regulatory T cell and an antibody-coated microparticle, providing external mechanical stimulation to a mixture, providing oscillatory stimulation to a mixture, stirring a mixture, rotating a mixture, providing an immunotherapy to a subject, treating a condition in a subject, providing activated immune cells to a subject, providing activated T cells to a subject, obtaining a biological sample from a subject, obtaining an immune cell from a subject, obtaining a T cell from a subject, obtaining peripheral blood mononuclear cells from a subject, generating activated immune cells, generating activated T cells, generating activated regulatory T cells, generating a microparticle, conjugating antibodies to a microparticle, and purifying antibody-coated microparticles. Any one or more of these steps may be excluded from embodiments of the present disclosure.

Disclosed herein, in some embodiments, is a method for activating an immune cell comprising (a) generating a mixture comprising (i) the immune cell and (ii) an antibody-coated microparticle; and (b) providing an external mechanical stimulation to the mixture. In some embodiments, the method further comprises, prior to (a), obtaining the immune cell from a subject. In some embodiments, the method further comprises, following (b), providing the immune cell to a subject. In some embodiments, the method further comprises providing to the subject an additional therapy. In some embodiments, the additional therapy is an immunotherapy. In some embodiments, the immune cell was obtained from the subject. In some embodiments, the immune cell was not obtained from the subject. In some embodiments, the subject suffers from or is suspected of having cancer. In some embodiments, the subject suffers from or is suspected of having a viral infection. In some embodiments, disclosed is a method for treating a subject for cancer, the method comprising (a) generating a mixture comprising (i) an immune cell and (ii) an antibody-coated microparticle; (b) providing an external mechanical stimulation to the mixture to generate activated immune cells from the immune cell; and (c) providing the activated immune cells to the subject.

In some embodiments, the immune cell is a T cell. In some embodiments, the T cell is a cytotoxic T cell. In some embodiments, the T cell is a CD4+ T cell. In some embodiments, the T cell is a CD8+ T cell. In some embodiments, the mechanical stimulation is an oscillatory stimulation. In some embodiments, the microparticle comprises at least 200 fg of antibodies, at least 500 fg of antibodies, or at least 750 fg of antibodies. In some embodiments, during (b), the immune cell expands at least 10-fold.

Disclosed herein, in some embodiments, is a method for activating a regulatory T cell (Treg), the method comprising generating a mixture comprising (i) the Treg and (ii) an antibody-coated microparticle comprising between 0.5 and 100 fg of antibodies. In some embodiments, the method further comprises, prior to (a), obtaining the Treg from a subject. In some embodiments, the method further comprises, following (b), providing the Treg to a subject. In some embodiments, the Treg was obtained from the subject. In some embodiments, the Treg was not obtained from the subject. In some embodiments, the subject suffers from or is suspected of having an autoimmune disorder. In some embodiments, disclosed is a method for treating a subject for an autoimmune disorder, the method comprising (a) generating a mixture comprising (i) a regulatory T cell (Treg) and (ii) an antibody-coated microparticle comprising between 0.5 and 100 fg of antibodies; (b) generating activated Tregs from the Treg; and (c) providing the activated immune cells to the subject. In some embodiments, the method further comprises providing an external mechanical stimulation to the mixture. In some embodiments, the mechanical stimulation is an oscillatory stimulation.

In some embodiments, the microparticle comprises between 1 and 50 fg of antibodies, between 15 and 40 fg of antibodies, or between 20 and 30 fg of antibodies. In some embodiments, the microparticle is greater than 4 μm in diameter and has an antibody density of less than 200 proteins per μm² on its surface. In some embodiments, the microparticle is less than 1 μm and has an antibody density of between 100 and 500 proteins per μm² on its surface. In some embodiments, the microparticle comprises one or more growth factors capable of stimulating a signaling pathway in the Treg. In some embodiments, the one or more growth factors are encapsulated within the microparticle. In some embodiments, the one or more growth factors comprise TGF-β or IL2. In some embodiments, during (b), the Treg expands at least 10-fold. In some embodiments, the Treg is an induced Treg.

In some embodiments, the oscillatory stimulation is provided at between 150 rotations per minute (rpm) and 500 rpm. In some embodiments, the oscillatory stimulation is provided at about 250 rpm. In some embodiments, the microparticle comprises antibodies or antibody-like particles. In some embodiments, the microparticle comprises anti-CD3 antibodies, anti-CD28 antibodies, anti-CD137 antibodies, or a combination thereof. In some embodiments, the microparticle comprises anti-CD3 and anti-CD28 antibodies.

In some embodiments, the microparticle has a stiffness of between 10 kPa and 30 kPa. In some embodiments, the microparticle has a stiffness of about 20 kPa. In some embodiments, the microparticle is between 0.2 μm and 5.0 μm in diameter. In some embodiments, the microparticle is an alginate microparticle. In some embodiments, the microparticle comprises a magnetic nanoparticle. In some embodiments, the magnetic nanoparticle is encapsulated within the microparticle. In some embodiments, the microparticle comprises a plurality of magnetic nanoparticles. In some embodiments, the external mechanical stimulation is provided for at least 12 hours, at least 24 hours, or at least 72 hours.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1F show data and details from studies described in Example 1. FIG. 1A shows a schematic representation of microfluidic generation of alginate microparticles encapsulating magnetic nanoparticles. FIG. 1B shows a size distribution analysis of prepared microparticles at different flow rates. FIG. 1C shows a size distribution analysis of selected particles (0.3 μm, left; 0.8 μm, middle; 4.5 μm, right). FIG. 1D shows the results from nanoindentation to assess the elastic modulus (lower curve, indentation; higher curve, retraction). FIG. 1E shows a schematic representation of proposed interactions between artificial antigen presenting cells and primary T cells. FIG. 1F shows a table with a summary of physical characteristics of prepared library of particles.

FIGS. 2A-2E show the distribution of antibodies conjugated to microparticles of the disclosure. FIG. 2A shows a confocal micrograph of microparticles coated with fluorescent anti-CD3 antibody. FIG. 2B shows a single slice through the equator of one of the particles FIG. 2C shows a false color image showing the intensity of fluorescence in the equatorial plane. FIG. 2D shows a quantification of fluorescence intensity in the equatorial plane. FIG. 2E shows the equatorial plane divided into two equal areas (“shells”) of 10.2 μm² and compared. On average, the outer shell shows 80.6% of the global integrated fluorescence intensity (n=20). The outer shell had an average pixel intensity of 37.1 while the inner shell had an average of 5.0.

FIGS. 3A-3E show results and details from studies described in Example 2. FIG. 3A shows a diagram demonstrating the co-culture of microparticles and T cells under static (left) or dynamic (right) conditions. FIG. 3B shows representative bright-field microscopy images of formed clusters by primary mouse T cells cultured with 4.5 μm aAPCs at a constant dose (1:1 particle/T-cell ratio) under static and dynamic (mechanical oscillation) cultures; scale bars=50 μm. FIG. 3C shows the mean volumes of T cells activated and expanded using various formulations of particles, as indicated, with the highest volume observed with “4.5” beads at a “1” antigen dose. FIG. 3D shows results from expansion of primary mouse CD4+ T cells by varying the antigen dose, particle size or the culture conditions after 4 days. FIG. 3E shows results from FACS quantification of CD8-to-CD4 ratio of T cells cultured with varying formulations of particles, compared to Dynabeads. The starting ratio for all conditions was 0.5.

FIGS. 4A-4C shows activation of T cells resulting in cellular enlargement (FIG. 4A), fold expansion in cell numbers (FIG. 4B), and change in the CD8 to CD4 ratio from a starting ration of 0.5 (FIG. 4C). Each dot represents an independent experiment. Horizontal line shows bootstrapped mean. Comparisons are made by permutation testing.

FIGS. 5A-5G show the results from proliferation and activation analyses of CD4+ T cells cultured under static or dynamic conditions in the presence of varying formulation of particles, as described in Example 3. FIG. 5A shows flow cytometry histograms of CFSE dilution of T cells after co-culturing with different formations of engineered microparticles in either dynamic or static conditions, as indicated. FIG. 5B shows percentage of proliferated T cells 3 days after co-culturing with different formations of engineered microparticles in either dynamic or static conditions, as indicated. FIGS. 5C and 5D show CD25 expression after 24 hours of co-culturing primary naive CD4+ T cells with 5 μm microparticles. FIG. 5E shows the percentage of CD25+ T cells 24 h after activation with various formulation of particles or Dynabeads. FIG. 5F shows CD44 expression histograms after 24 h of co-culturing of primary naive CD4+ T cells with 5 um microparticles presenting various surface densities of antibodies under static or dynamic culture. FIG. 5G shows the percentage of CD44+ T cells 24 h after activation with various formulation of particles or Dynabeads.

FIGS. 6A-6C show the proliferation of T cells measured by CFSE dilution and evaluated by FlowJo for percent proliferated (FIG. 6A), division index (FIG. 6B), and proliferation index (FIG. 6C). Each dot represents an independent experiment. Horizontal line shows bootstrapped mean. Comparisons are made by permutation testing.

FIGS. 7A-7B show expression of CD25+ (FIG. 7A) and CD44+ (FIG. 7B) T cells after co-culture with aAPCs of various sizes and antibody conjugation densities. Each dot represents an independent experiment. Horizontal line shows bootstrapped mean. Comparisons are made by permutation testing.

FIGS. 8A-8B shows the results from the immune synapse size studies described in Example 4. FIG. 8A shows a confocal microscopy image of immune synapses formed by OT-II T cells activated with 4.5 μm (“1”) microparticles interacting with (antigen-pulsed) antigen presenting cells (B lymphoma cells). Images show overlap of confocal slices. Representative cells that had the median immune synapse volume were chosen. FIG. 8B shows a graph of an analysis of immune synapse volumes (in μm³) formed by primary naive T cells activated with various particle sizes with high or low antibody conjugation level cultured under static or dynamic conditions. Each dot represents an immune synapse between a T cell and an antigen presenting cell (n=16 per condition). Boxes show means and 95% CI values. Results are representative of three independent experiments.

FIG. 9 is a graph showing the release of TGF-β and IL-2 from alginate-heparin microparticles at 37° C.

FIGS. 10A-10D show results from flow cytometric analysis of iTreg development assessed by flow cytometry for Foxp3 and CD25 coexpression after co-culture of naive CD4+ T-cells with particles at various formulations either under dynamic or static conditions for 4 days. FIGS. 10A and 10B show the percentage of induced Tregs (FIG. 10A) and mean fluorescence intensity (MFI) of Foxp3 expression in T cells (FIG. 10B) 4 d after activation with various formulation of particles or Dynabeads. FIG. 10C shows the stability of formed T-regs as assessed by measuring the change in the population of iTregs (T cells expressing CD4, CD25, and Foxp3+) after 4 and 8 days. FIG. 10D shows results from a T-cell suppression assay; flow sorted Tregs were co-cultured with naive primary CD4+ T-cells (Tconv) at three different ratios of cell counts (1:1, 1:10, and 1:30 of Treg to Tconv) in the presence of surface-coated anti-CD3 and soluble anti-CD28 for 3 days.

FIGS. 11A-11B demonstrate the relationship between the number of antibody molecules on the disclosed antibody-coated microparticles and the development of Tregs (FIG. 11A) or activation of conventional T cells (FIG. 11B).

FIG. 12 shows a graph of % T cell activation versus oscillatory agitation speed (rpm).

### DETAILED DESCRIPTION OF THE INVENTION

Ex vivo cultivation of T cells is important for manufacturing cellular therapies, such as chimeric antigen receptor (CAR)-T cells. Therefore, the optimization of approaches for polyclonal T-cell cultivation is clinically important.¹ In the activation of T cells for biomedical engineering applications, such T cells may be cultured with beads coated with stimulatory antibodies, or artificial antigen presenting cells (aAPCs). aAPCs have been developed using microtechnology and nanotechnology approaches, developing particles that can be co-cultured with T cells or engineered surfaces that offer stimulatory signals.⁸ The

In certain embodiments, to determine the effect of strength of stimulatory signals on T-cell activation, the inventors fabricated aAPC microparticles and conjugated anti-CD3 and anti-CD28 antibodies at different densities. To test the effect of various degrees of curvature and different surface areas of contact with T cells, the aAPCs comprised spheres of different sizes. In some embodiments, to test whether an external mechanical stimulus upon the TCR could promote activation, the inventors also engaged an oscillatory movement to the aAPCs. Aspects of the present disclosure provide methods and compositions that dramatically improve activation beyond conventional stimulation. In another example, disclosed are aAPC conditions that offer a “sweet spot” of signaling to maximize the production of induced regulatory T cells (iTreg), the development of which is actually hindered by high levels of stimulation. In some examples, the microparticles were also endowed with the ability to secrete cytokines to further promote iTreg development.

### I. Microparticles

Aspects of the disclosure provide microparticles useful for immune cell activation. In some embodiments, provided herein are antibody-coated microparticles capable of activating immune cells (e.g., T cells). As used herein, “antibody-coated microparticles” describe microparticles having attached (on the surface and/or internally) one or more antibodies or antibody-like molecules (e.g., antibody fragments, scFv molecules, etc.). In some embodiments, antibody-coated microparticles comprise one or more antibodies capable of T cell activation. Examples of antibodies capable of T cell activation include anti-CD3, anti-CD28, and anti-CD137 antibodies.

Microparticles may comprise antibodies at various densities. The density of antibodies may describe an amount of antibodies per microparticle in a given population of microparticles. Microparticles of the present disclosure may comprise at least or at most 0.5, 1, 2, 3, 4, 5, 10, 15, 20, 25, 30, 35, 40, 45, or 50 fg of antibodies (or antibody-like molecules) per microparticle (or any range derivable therein). In some embodiments, microparticles comprise 1-50, 5-50, 10-50, 15-40, 20-30, or 25-30 fg of antibodies per microparticle, or any range or value derivable therein. Microparticles of the present disclosure may comprise at least 100, 200, 300, 400, 500, 600, 700, or 800 fg of antibodies per microparticle, or any range or value derivable therein. In some embodiments, the disclosed microparticles comprise about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 antibodies (or antibody-like molecules) per microparticle, or any range or value derivable therein.

Microparticles of the present disclosure may comprise various materials, which may be selected depending on the desired properties. In some embodiments, microparticles comprise alginate. In some embodiments, microparticles comprise polystyrene. In some embodiments, microparticles comprise one or more magnetic nanoparticles (e.g., superparamagnetic iron oxide nanoparticles). Microparticles may comprise one or more proteins. In some embodiments, microparticles comprise one or more growth factors for induction of regulatory T cells (Tregs). Examples of such growth factors include TGF-β and IL2.

In some aspects, the disclosed microparticles are generated using microfluidic techniques. In one example, a microfluidic generator is used to encapsulate a polymer and, in some cases, one or more magnetic nanoparticles within a microfluidic droplet. A cross-linker may be provided to stimulate cross-linking and bead formation within the droplet. Various properties of the microparticle may be modified by modifying the microfluidic conditions. For example, the sheath flow may be adjusted to tune the size of the microparticles to a desired value. In some embodiments, the disclosed microparticles are between about 150 nm and 10 μm in size. In some embodiments, the microparticles are 150 nm-10 μm, 300 nm-10 μm, 500 nm-10 μm, 1 μm-10 μm, 5 μm-10 μm, 150 nm-5 μm, 150 nm-1 μm, 150 nm-500 μm, or any value or range derivable therein. In some embodiments, the microparticles are at least, at most, or about 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, or 10000 nm in size, or any range or value derivable therein. In some embodiments, the microparticles are at least, at most, or about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 μm in size, or any range or value derivable therein. In some embodiments, the microparticles are at least 300 nm in size. In some embodiments, the microparticles are at least 500 nm in size. In some embodiments, the microparticles are at most 10 μm in size. In some embodiments, the microparticles are about 0.3 μm in size. In some embodiments, the microparticles are about 0.8 μm in size. In some embodiments, the microparticles are about 4.5 μm in size.

In some embodiments, the disclosed microparticles are of a given stiffness. A stiffness of a microparticle may be adjusted to maximize spreading of an immune cell (e.g., T cell) on a microparticle, thereby maximizing activation of the immune cell. In some embodiments, a microparticle has a stiffness of at least, at most, or about 5, 6, 7, 8, 9, 10, 11, 12, 13, 14, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 kPa, or any range or value derivable therein. In some embodiments, the microparticle has a stiffness of between 10 kPa and 30 kPa, or any range or value derivable therein. In some embodiments, the microparticle has a stiffness of about 20 kPa. In some embodiments, the microparticle has a stiffness of about 15 kPa. In some embodiments, the microparticle has a stiffness of about 14.6 kPa.

A property of a population of microparticles (e.g., size, stiffness, antibody density) may be provided as an “average”, such that the microparticles of the population have a certain property “on average”. In these cases, the value of the property is provided as the average of all microparticles in the population, recognizing that multiple microparticles in the population may have different values for the property.

### II. Immune Cells

Aspects of the present disclosure comprise activation of immune cells. Activation of an immune cell may comprise stimulating cellular growth and division. In some embodiments, disclosed herein is activation of T cells using stimulatory molecules (e.g., antibodies). T cell activation may be useful in, for example, expansion of therapeutic T cells for treatment methods. Examples of therapeutic T cells which may be activated using the compositions and methods of the present disclosure include engineered T cells (e.g., CAR T cells) and isolated, natural T cells (e.g., tumor-associated lymphocytes). T cell activation may also be useful in, for example, expansion of T cells for research or diagnostic purposes.

T cells may be obtained from any suitable source for use with the compositions and methods of the present disclosure. In some embodiments, T cells are obtained from a biological sample from a subject. A biological sample may be a blood sample

Various types of T cells may be activated using the disclosed methods and compositions. In some embodiments, methods of the present disclosure comprise activation of conventional T cells. Examples of conventional T cells include CD4+ T cells and CD8+ T cells. In some embodiments, the present disclosure comprises activation of cytotoxic T cells. In some embodiments, the present disclosure comprises activation of regulatory T cells (Tregs). Tregs may be natural Tregs or induced Tregs (iTregs). Treg activation may comprise generation of induced Tregs from conventional T cells. Induced Tregs may describe T cells generated by stimulation of T cells with appropriate growth factors, for example IL2 and/or TGF-β. Examples of generation of iTregs are described in further detail in, for example, Majedi, F. S., et al., Adv. Mater. 2018, 30, 1703178, incorporated herein by reference in its entirety.

In some embodiments, T cell activation comprises providing to a T cell an antibody-coated microparticle of the present disclosure (e.g., a microparticle comprising anti-CD3, anti-CD28, and/or anti-CD137 antibodies). In some embodiments, T cell activation comprises mechanical stimulation (e.g., oscillatory stimulation). Mechanical stimulation may be useful for increasing the activation strength for conventional or cytotoxic T cells. In some embodiments, T cell activation does not comprise mechanical stimulation. For example, in some embodiments, activation of regulatory T cells does not comprise mechanical stimulation.

### III. Mechanical Stimulation

Aspects of the present disclosure comprise mechanical stimulation of a mixture comprising microparticles and immune cells. Mechanical stimulation may describe providing external forces (i.e., forces requiring an external energy source such as electrical or mechanical energy) to a mixture to generate movement of the microparticles and/or immune cells within the mixture. Examples of mechanical stimulation include agitation, sonication, vibration, and the like. In some embodiments, mechanical stimulation is oscillatory stimulation. Examples of oscillatory stimulation include shaking, rotating, spinning, and the like. In some embodiments, oscillatory stimulation of the present disclosure comprises shaking of a mixture comprising a microparticle and an immune cell. Mechanical stimulation may be provided by a suitable source, such as, for example, a mechanical rocker, an orbital shaker, a mechanical rotator, manual stirring, automated stirring, and the like. In some embodiments, oscillatory stimulation is provided by an orbital shaker. In some embodiments, oscillatory stimulation is provided by a mechanical rotator.

In some embodiments, a mixture comprising a microparticle and an immune cell is provided oscillatory stimulation at between 150 and 400 rotations per minute (rpm), or any range or value derivable therein. In some embodiments, the oscillatory stimulation is at least, at most, or about 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 rpm, or any range or value derivable therein. In some embodiments, the oscillatory stimulation is about 250 rpm. In some embodiments, the oscillatory stimulation is about 240 rpm.

In some embodiments, mechanical stimulation is provided to a mixture for a particular duration of time. In some embodiments, mechanical stimulation is provided for between 1 and 48 hours, or any range or value derivable therein. In some embodiments, mechanical stimulation is provided for at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours, or any range or value derivable therein. In some embodiments, mechanical stimulation is provided for 1-48, 4-48, 12-48, 24-48, 36-48, 1-36, 1-24, 1-18, 4-18, 6-18, or 10-18 hours, or any range or value derivable therein. In some embodiments, mechanical stimulation is provided for about 8 hours. In some embodiments, mechanical stimulation is provided for about 12 hours. In some embodiments, mechanical stimulation is provided for about 16 hours. In some embodiments, mechanical stimulation is provided for about 24 hours.

### IV. Therapeutic Methods

The compositions of the disclosure may be used for in vivo, in vitro, or ex vivo administration. The route of administration of the composition may be, for example, intracutaneous, subcutaneous, intravenous, local, topical, and intraperitoneal administrations.

In some embodiments, therapeutic methods of the present disclosure comprise treatment or prevention of cancer. For example, in some embodiments, T cells (e.g., cytotoxic T cells) are expanded and provided to a subject to treat cancer. The cancer may be a solid tumor, metastatic cancer, or non-metastatic cancer. In certain embodiments, the cancer may originate in the bladder, blood, bone, bone marrow, brain, breast, urinary, cervix, esophagus, duodenum, small intestine, large intestine, colon, rectum, anus, gum, head, kidney, liver, lung, nasopharynx, neck, ovary, prostate, skin, stomach, testis, tongue, or uterus. In some embodiments, the cancer originates in the colon. In some embodiments, the cancer originates in the rectum.

The cancer may specifically be of one or more of the following histological types, though it is not limited to these: undifferneiated carcinoma, bladder, blood, bone, brain, breast, urinary, esophageal, thymomas, duodenum, colon, rectal, anal, gum, head, kidney, soft tissue, liver, lung, nasopharynx, neck, ovary, prostate, skin, stomach, testicular, tongue, uterine, thymic, cutaneous squamous-cell, noncolorectal gastrointestinal, colorectal, melanoma, Merkel-cell, renal-cell, cervical, hepatocellular, urothelial, non-small cell lung, head and neck, endometrial, esophagogastric, small-cell lung mesothelioma, ovarian, esophogogastric, glioblastoma, adrencorical, vueal, pancreatic, germ-cell, giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; thymoma; thecoma; androblastoma; sertoli cell carcinoma; malignant melanoma; amelanotic melanoma; superficial spreading melanoma; epithelioid cell melanoma; sarcoma; mesenchymal (e.g., fibrosarcoma; fibrous histiocytoma; myxosarcoma; liposarcoma; leiomyosarcoma; rhabdomyosarcoma; embryonal rhabdomyosarcoma; alveolar rhabdomyosarcoma; stromal sarcoma; mullerian mixed tumor; nephroblastoma; hepatoblastoma; carcinosarcoma; dysgerminoma; embryonal carcinoma; choriocarcinoma; mesonephroma; hemangiosarcoma; Kaposi's sarcoma; lymphangiosarcoma; osteosarcoma; juxtacortical osteosarcoma; chondrosarcoma; mesenchymal chondrosarcoma; giant cell tumor of bone; Ewing's sarcoma; ameloblastic odontosarcoma; ameloblastic fibrosarcoma; chordoma; ependymoma; astrocytoma; protoplasmic astrocytoma; fibrillary astrocytoma; astroblastoma; oligodendroglioma; oligodendroblastoma; primitive neuroectodermal; cerebellar sarcoma; ganglioneuroblastoma; neuroblastoma; retinoblastoma; olfactory neurogenic tumor; neurofibrosarcoma; paragranuloma); or hematopoietic (e.g., multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia).

In some embodiments, therapeutic methods of the present disclosure comprise treatment or prevention of an autoimmune or inflammatory condition. For example, in some embodiments, Tregs are expanded and provided to a subject to treat an autoimmune or inflammatory condition. The autoimmune condition or inflammatory condition amenable for treatment may include, but not be limited to conditions such as diabetes (e.g. type 1 diabetes), graft rejection, arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and systemic juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, Lichen nitidus, Lichen sclerosus et atrophicus, Lichen simplex chronicus, Lichen spinulosus, Lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, juvenile onset (Type I) diabetes mellitus, including pediatric insulin-dependent diabetes mellitus (IDDM), and adult onset diabetes mellitus (Type II diabetes) and autoimmune diabetes. Also contemplated are immune responses associated with acute and delayed hypersensitivity mediated by cytokines and T-lymphocytes, sarcoidosis, granulomatosis including lymphomatoid granulomatosis, Wegener's granulomatosis, agranulocytosis, vasculitides, including vasculitis, large-vessel vasculitis (including polymyalgia rheumatica and gianT cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), Addison's disease, autoimmune neutropenia, pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including Pemphigus vulgaris, Pemphigus foliaceus, pemphigus mucus-membrane pemphigoid, and Pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, autoimmune or immune-mediated thrombocytopenia such as idiopathic thrombocytopenic purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), experimental autoimmune encephalomyelitis, myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, gianT cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia greata, alopecia totalis, CREST syndrome (calcinosis, Raynaud's phenomenon, esophageal dysmotility, sclerodactyl), and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, gianT cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, asperniogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, graft versus host disease, contact hypersensitivity, asthmatic airway hyperreaction, and endometriosis.

### V. Immunotherapy

In some embodiments, the methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immumotherapies are known in the art, and some are described below.

1. Inhibition of Co-Stimulatory Molecules

In some embodiments, the immunotherapy comprises an inhibitor of a co-stimulatory molecule. In some embodiments, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

A. Checkpoint Inhibitors and Combination Treatment

Embodiments of the disclosure may include administration of immune checkpoint inhibitors, examples of which are further described below.

1. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

2. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO0 1/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

B. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

C. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some embodiments, the CAR-T therapy targets CD19.

D. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

E. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein.

### VI. Sample Preparation

In certain aspects, methods involve obtaining a sample (also “biological sample”) from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In certain embodiments the sample is obtained from a biopsy from esophageal tissue by any of the biopsy methods previously mentioned. In other embodiments the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples, such as multiple esophageal samples may be obtained for diagnosis by the methods described herein. In other cases, multiple samples, such as one or more samples from one tissue type (for example esophagus) and one or more samples from another specimen (for example serum) may be obtained for diagnosis by the methods. In some cases, multiple samples such as one or more samples from one tissue type (e.g. esophagus) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one embodiment, the sample is a fine needle aspirate of a esophageal or a suspected esophageal tumor or neoplasm. In some cases, the fine needle aspirate sampling procedure may be guided by the use of an ultrasound, X-ray, or other imaging device.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Generation of Antibody-Coated Microparticles

Results

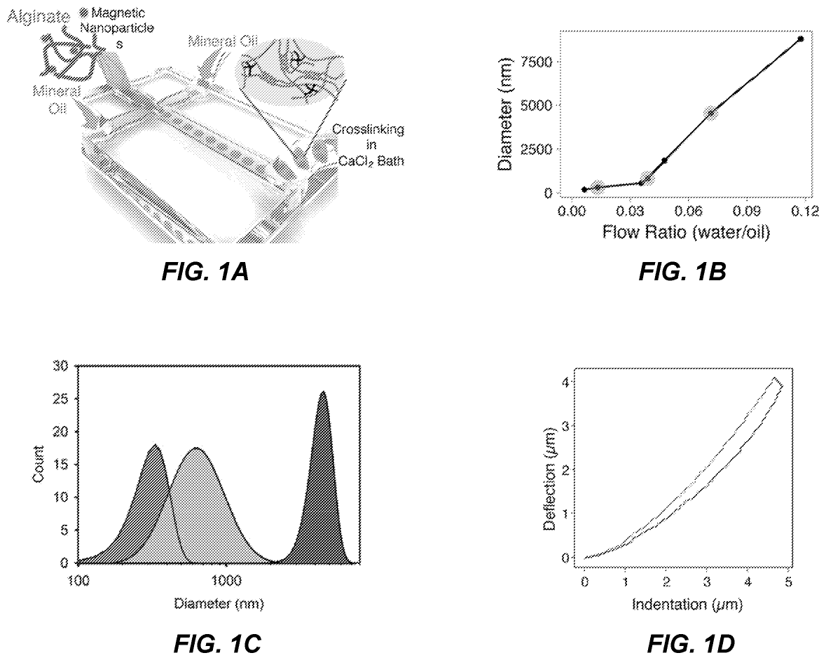

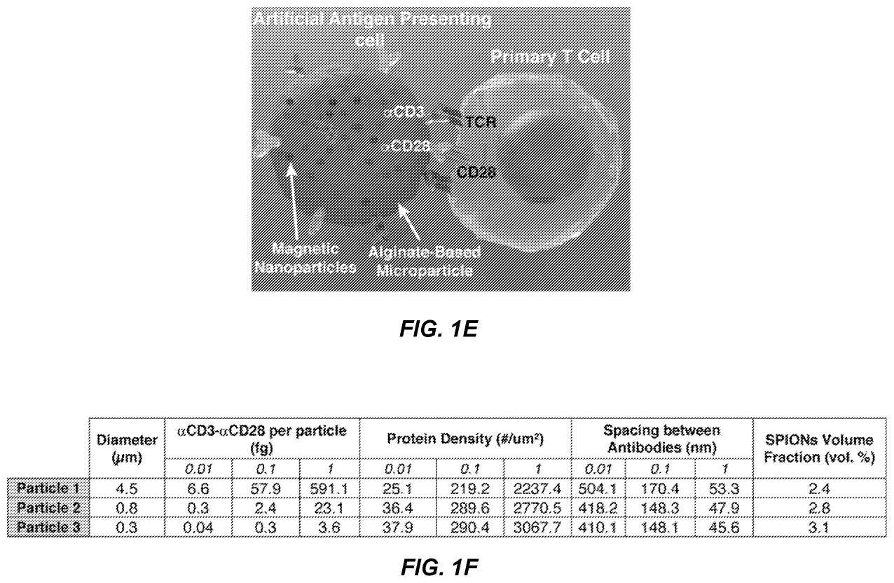

To develop the antibody-coated microparticles (“artificial APCs”), a microfluidic droplet generator was utilized capable of encapsulating alginate polymer and magnetic nanoparticles (FIG. 1A). A constant fraction of cross-linker (4-arm PEG hydrazide) was mixed with the alginate polymer in the main channel. During droplet formation, magnetic nanoparticles were encapsulated within our beads magnetic nanoparticles that were 100-nm-diameter, carboxylated super paramagnetic iron oxide nanoparticles (SPIONs). The microfluidic approach produced a homogeneous collection of particles, as verified by dynamic light scattering (FIG. 1C). To tune the size of particles ranging from 150 nm to 10 μm, the ratio of central to sheath flow was varied (FIG. 1B). To employ a broad range of particle sizes in our experiments (over one order of magnitude), three sizes of particles were selected with average radii of 307 nm (“0.3”), 824 nm (“0.8”), and 4540 nm (“4.5 μm”, or “5 μm”) (FIG. 1C). In pilot work, it was found that nanoparticles of diameters in the range 50-250 nm could not be fully separated from each other or from T cells due to partial internalization or trapping on the rough cell surface, whereas particles larger than 300 nm could be separated from cells with >95% efficiency. The resulting alginate particles were then collected in a bath of 200 mM CaCl2, followed by a ˜40 min incubation to reach complete gelation. Eventually particles were subjected to overnight chemical cross-linking through the hydrazine linker. Excess calcium and cross-linker were removed by serial washing with phosphate buffered saline (PBS). The mechanical stiffness of these aAPC microparticles was measured by nanoindentation and found to be 14.6 kPa (FIG. 1D), a substrate stiffness that allowed for maximal spreading of T cells.

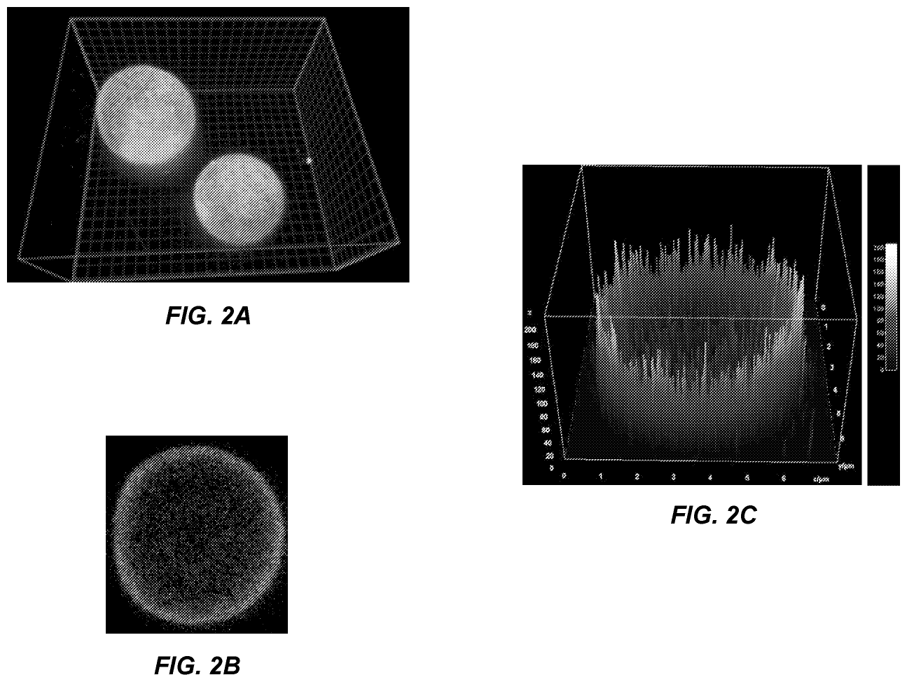

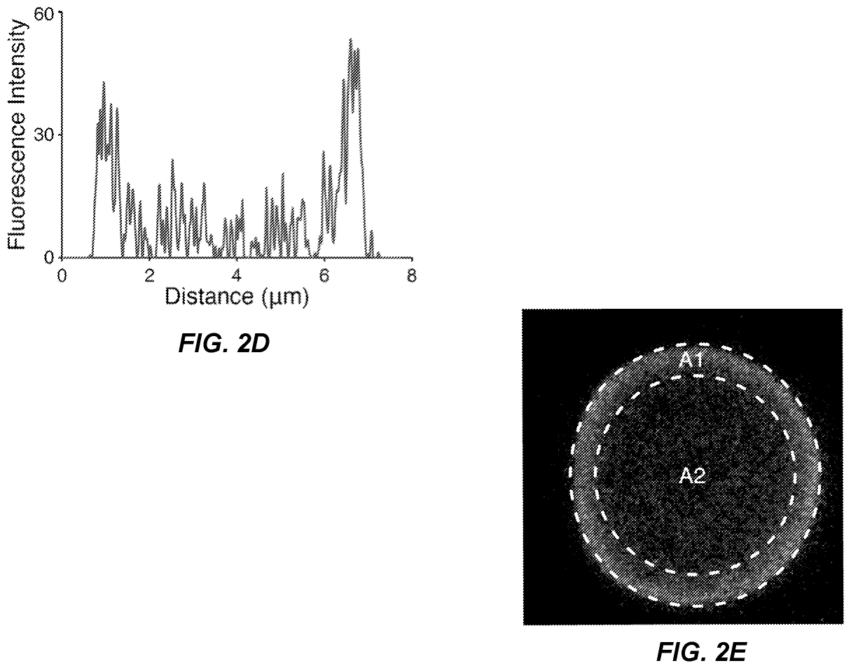

Next, stimulatory antibodies were conjugated to the surface of the microparticles. The carboxylic groups of alginate provide a versatile platform for antibody conjugation. Using NHS/EDC chemistry, anti-CD3 and anti-CD28 antibodies were conjugated (see the Methods section below) and washed away excess antibodies and quenching unreacted groups through repeated washing with phosphate buffered saline solution containing 0.5% w/v BSA. To characterize the conjugation of antibodies, they were imaged by confocal microscopy and found that over 80% of antibodies were conjugated to the outside the particles (as shown in FIGS. 2A-2E). Based on pilot experiments, three different densities of antibodies were chosen, representing 10-fold dilutions, to coat beads representing high (“1”), medium (“0.1”), and low (“0.01”) amounts of antigenic signaling. An average of 2692±420, 266±41, and 33±7 antibody molecules per square micrometer were immobilized as high, medium, and low conjugation densities, respectively (FIG. 1F). For comparison, a theoretical limit of ˜12 732 antibodies could be packed into a square micrometer, assuming that an antibody has a radius of ˜5 nm.² Thus, the “high” labeling density corresponds to ˜21% of the theoretical limit. The size of the interface between T cells and antigen presenting cells (FIG. 1E) varies based on cytoskeletal state of the T cell,³ with most contacts falling in the range of 5-25 μm². If an area of 10 μm² is assumed for a typical immune synapse, the large particles (2.25 μm radius) would offer a hemispheric area of ˜32 μm², so that an immune synapse-sized 10 μm² would engage ˜⅓ of the hemisphere and would engage ˜22 000 antibodies (high density conjugation), 2192 antibodies (medium), or 251 antibodies (low). For the medium-sized particles (0.3 μm radius), a hemispheric immune synapse offers an area of ˜1 μm² and 2785, 291, and 36 molecules, at these respective conjugation densities (high, medium, low). For the smallest particles (0.15 μm radius), a hemispheric immune synapse would engage 433, 41, and 5 molecules, respectively. The experimentally observed minimum amount of signaling needed to activate a T cell ranges from 1 to 4 engaged TCRs.^{4,5} Thus, in all cases, the number of T-cell receptors the microparticles can engage in the immune synapse should exceed that minimum.

Methods

The alginate was charcoal treated and sterile filtered (0.22 μm filters, Millipore, Billerica, Mass.) prior to the particle formation. A hydrophobic glass microfluidic droplet junction chip (channel depth 100 μm; Dolomite Microfluidics, Charlestown, Mass.) was utilized to make monodispersed hydrogel droplets as microparticle substrates. A mixture of alginate solution (1% w/v) and 4-arm PEG hydrazide (MW 5 kDa, Creative PEG work, Chapel Hill, N.C.) (5 mM) was used as the inner aqueous phase. Mineral oil containing 10 wt % surfactant Span 80 was used as the continuous phase. Several flow rates were applied using two syringe pumps (Fusion 200, Chemyx, Stafford, Tex.) for the alginate and oil flows to control formation of alginate-based droplets. Images were taken at various time points using a Leica DMIL inverted fluorescence microscope fitted with appropriate filters and connected to a camera. Once particles were formed, they were collected in a bath containing calcium ions (100 mM CaCl2) and left at room temperature for 45 min for ionic crosslinking. The microgels were extensively washed with 10 mM NaCl solution and centrifuged (15,000 rpm for 10 min) twice before further incubation in a solution containing hydroxybenzotriazole (HOBt) and 1-ethyl-3-(3-dimethylaminopropyl)carbodi-imide (EDC). After 2 h, particles were dialyzed against deionized water for three days extensively to remove any residual reagents, then frozen at −20° C. and lyophilized. Particles were then resuspended either in deionized water or phosphate-buffered saline (PBS) for further use. Magnetic microparticles were fabricated with the addition of super paramagnetic iron oxide nanoparticles (SPION; 50 nm, carboxylated, Chemicell GmbH, Berlin, Germany) to the alginate/PEG mixture. The solution was (bath) sonicated for 10 min at 4° C. prior to use.

For the preparation of antibody-conjugated alginate particles, EDC/NHS chemistry was used to covalently conjugate anti-CD3 (2C11; Bio X Cell) and anti-CD28 (37.51; Bio X Cell) to the surface of particles. After activation of particles' carboxylic groups for 10 min and washing them with PBS (1×) twice, these proteins were added to the particles and vortexed briefly before stirring overnight at 4° C. The protein-functionalized microparticles were then magnetically separated from unbound proteins and washed several times with PBS (1×). Unreacted functional groups were quenched by washing samples in Tris buffer (100 mM, pH 8).

An antibody density of 10 μg/mL was used as the “high” amount of antibody (“1” in FIG. 1E). Dilutions of 10- and 100-fold were made as “medium” (or “moderate”) and “low” conjugation densities. The number of particles of different sizes employed in stimulation experiments were adjusted so that their total surface areas were equivalent across conditions order to provide the same surface area for protein conjugation and also for co-culturing with T cells. The amount of antibody (protein) that was used is summarized in Table 3.

Quantification of the total amount of anti-CD3 and anti-CD28 presented on functionalized particles was analyzed using micro-BCA assay according to the manufacturer's protocol. Dynamic light scattering (DLS) and zeta potential measurements were performed using a Zetasizer (Zetasizer 3000HS, Malvern Instruments Ltd., Worcestershire, UK) in backscattering mode at 173° for the diluted suspensions in water. The iron content of the particles was measured by ICP-OES (Inductively Coupled Plasma—Optical Emission Spectrometry) after digestion with alginate lyase.

Mechanical properties of microparticles were measured using Piuma Nanoindenter (Optics 11, Netherlands) with indentation performed by latex beads fixed to the end of cantilevers. The spring constant of the cantilever used in this work was 0.47 N/m. Measurements were performed in liquid mode (ultrapure water) at room temperature. The force-displacement curves were recorded with the vertical ramp size of 10 μm.

TABLE 1
Evaluation of super paramagnetic iron oxide nanoparticles inside the alginate microparticles

| | | | | | Average | Volume | Distribu |
|---|---|---|---|---|---|---|---|
| | | | | SPION | Number of | fraction of | tion |
| | Diameter | Volume | Relative | Weight | SPIONs per | SPIONs | Density |
| | (nm) | (nm3) | Volume | Content | Particles | (vol. %) | (nm3) |
| Particle 3 | 307 | 1.5 × 10⁷ | 1 | 0.25 | 5.65 | 2.43 | 3.73 × 107 |
| Particle 2 | 824 | 2.9 × 10⁸ | 19.3 | 0.33 | 126.39 | 2.82 | 4.31 × 107 |
| Particle 1 | 4540 | 4.9 × 10¹⁰ | 3234.1 | 0.36 | 22934.98 | 3.06 | 4.68 × 107 |

TABLE 2
Relative numbers of microparticles used in co-culture studies

| | | Surface Area/ | Total Required | Number of Particles | Relative |
|---|---|---|---|---|---|
| | Diameter | Particle | Surface Area | to Provide | Number of |
| | (nm) | (μm2) | (μm2) | 1 Particle:1 Cell | Particles |
| Particle 1 | 4.5 | 63.62 | 9.5 × 107 | 1.5 × 106 | 1 |
| Particle 2 | 0.8 | 2.01 | 9.5 × 107 | 4.7 × 107 | 31.64 |
| Particle 3 | 0.3 | 0.28 | 9.5 × 107 | 3.4 × 108 | 225 |
| Dynabead | 4.5 | 63.62 | 9.5 × 107 | 1.5 × 106 | 1 |

TABLE 3
Antibody (protein) used to coat microparticles

| | | | Required | | | | | Requir |
|---|---|---|---|---|---|---|---|---|
| | | Surface | total protein | Number | Total | Total | Particle Stock | ed # |
| | Diameter | area | per particle | particles per | aCD3 | aCD28 | Concentration | of |
| | (μm) | (μm2) | (fg) | test* | (μg) | (μg) | (#/mL) | particles |
| Particle 1 | 4.5 | 63.62 | 795.19 | 1.5 × 106 | 1.19 | 0.30 | 150 × 106 | 1.5 × 106 |
| Particle 2 | 0.8 | 2.01 | 25.13 | 51.9 × 106 | 1.30 | 0.33 | 25 × 109 | 1.6 × 109 |
| Particle 3 | 0.3 | 0.28 | 3.53 | 193.7 × 106 | 0.68 | 0.17 | 220 × 109 | 44 × 109 |

*per 1.5 × 10⁶ T cell

### Example 2—Assessment of the Impact of Mechanical Stimulation on T Cell Activation with Antibody-Coated Microparticles

Results

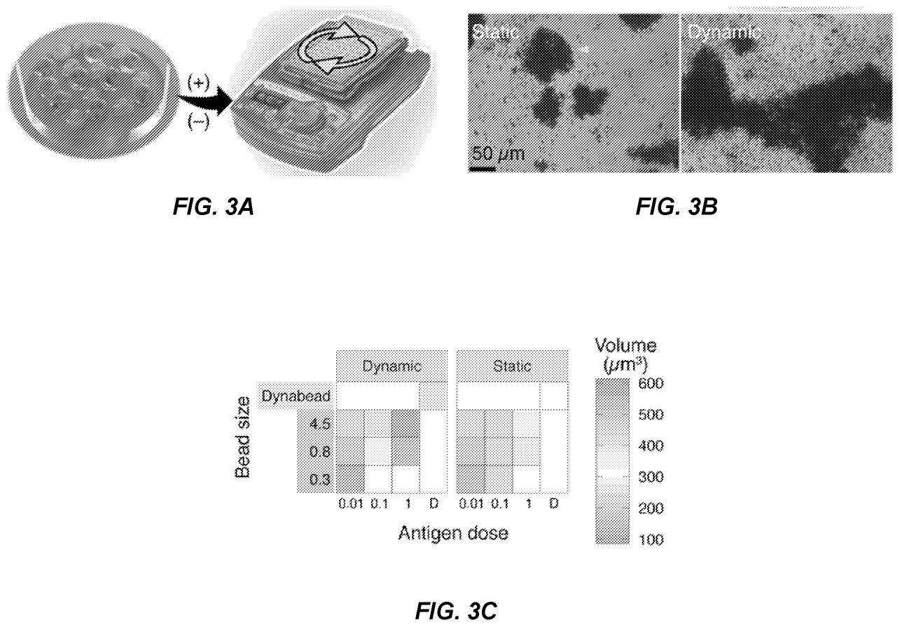

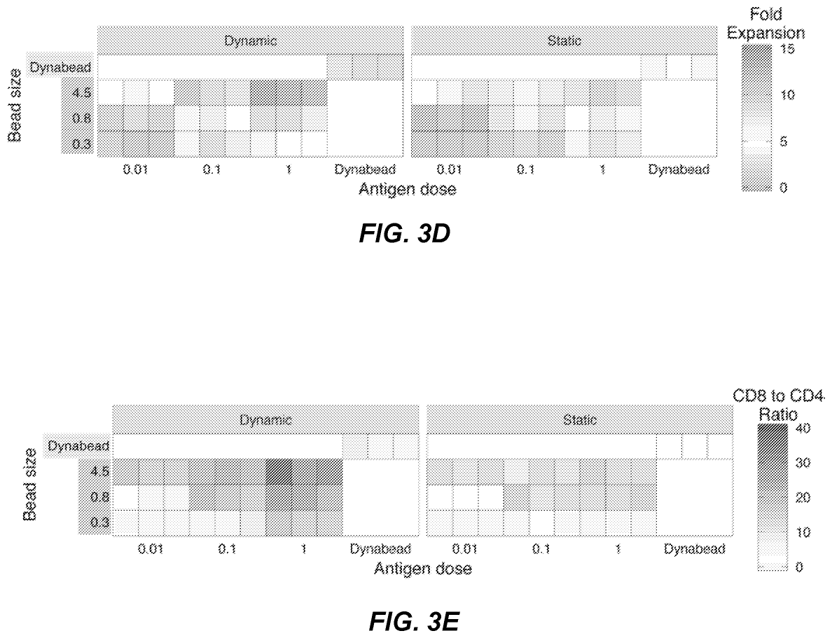

To assess the impact of external, gentle mechanical stimulation on co-cultures of T cells with aAPCs, an orbital shaker was used to deliver a continuous oscillatory movement of either ˜250 rpm rotational speed (“dynamic”) or switched off (“static”) (FIG. 3A). The impact of the aAPCs on T cells was compared with that of Dynabeads™ CD3/CD28 T-cell expansion beads (Life Technologies). Primary T cells were obtained from spleens of wild-type mice, enriched by negative magnetic-bead selection, and cultured with aAPCs under either static or dynamic conditions.

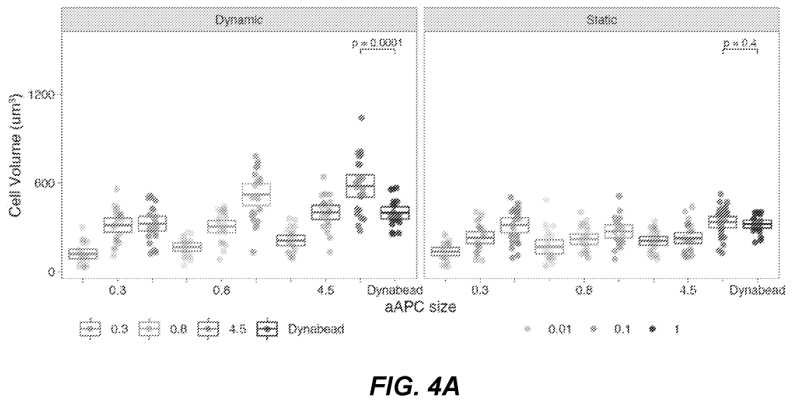

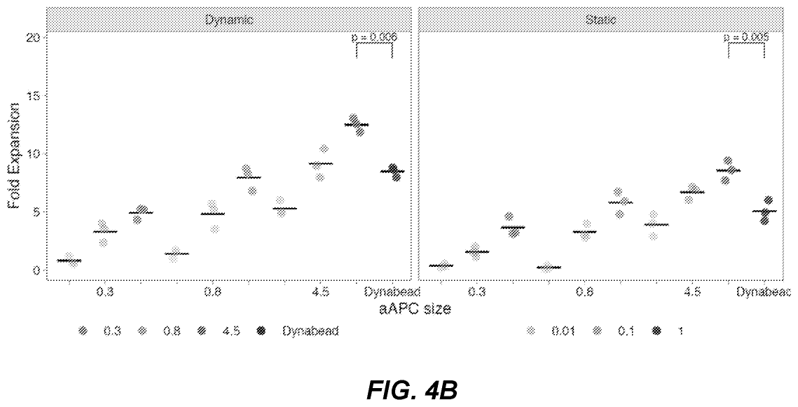

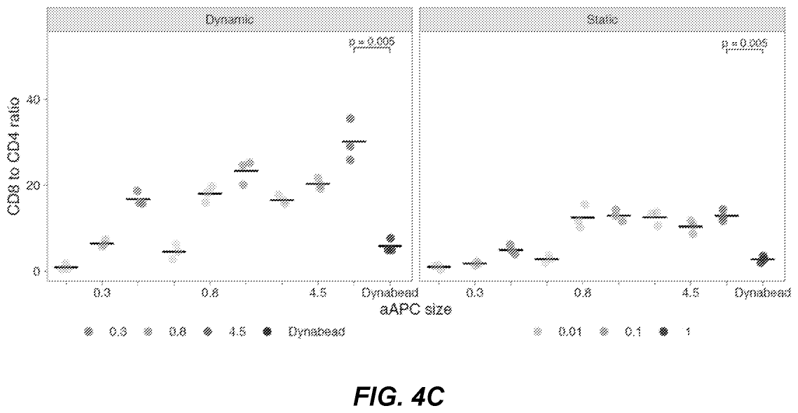

By day 2 or 3 of culture, polyclonal, primary mouse T cells formed large clusters with the beads. The clusters were obviously larger in the dynamic culture than in static culture (shown in FIG. 3B). The T cells were separated from the beads and imaged by 3D confocal microscopy to assess their growth. Cell volume was quantified since the volume changes as a cell grows, proliferates, or differentiates. Cell growth was larger in dynamic culture versus static culture across all particle sizes and conjugation densities (FIG. 3C; statistical comparisons are shown in FIG. 4A). The average volume of T cells co-cultured with Dynabeads under static conditions (n=22) was 321±26 μm³ (mean, ±95% CI), which represents an average diameter of 8.5±0.34 μm. The largest cells were those resulting from co-culture with 4.5 μm particles at the highest density of ligands, which had average volume of 580±74 μm³, corresponding to a diameter of 10.3±0.82 μm (n=25).

To assess their proliferative response, T cells were counted after 3 days of co-culture with the various particles. Under all conditions, dynamic culture resulted in significantly higher expansion of T cells than static culture (FIG. 3D; statistical comparisons are given in FIG. 4B). The average fold expansion of T cells co-cultured with Dynabeads under static conditions (n=3) was (5±1.8)-fold (mean, ±95% CI). T cells proliferated much more in culture with the disclosed mechanically soft particles of the same size and antibody loading as Dynabeads than with Dynabeads, suggesting that the softer mechanics of the disclosed microparticles offers an additional stimulus for activation and proliferation ((8.6±1.8)-fold expansion, p=0.006, compared to static Dynabeads). The largest expansion of T-cell count was observed under conditions where T cells were cultured in oscillating conditions with the 4.5 μm microparticles at the high density of stimulatory antibodies, resulting in an increase of 12.5±1.2 fold (p=0.004, compared to static Dynabeads). Averaging across all particle sizes and antigen doses, mechanical oscillation increased the proliferation of the cells by 2.0-fold, compared to static culture (ANOVA considering movement, size, and dose; movement p=1.5×10⁻¹²).

Generally, cytotoxic CD8+ T cells have a higher proliferative capacity than CD4+ T cells. Cytotoxic T cells have important applications in engineered cancer immunotherapies. The ability of these particles to promote cytotoxic T-cell expansion was assessed by monitoring the CD8-to-CD4 T-cell ratio during proliferation. CD4+ T cells and CD8+ T cells were separately purified from mice, then mixed them to achieve the physiological ratio of one CD8+ T cell to two CD4+ T cells. The T cells were co-cultured with particles as above, and, after 5 days, the ratio of CD8 to CD4 T cells was measured by flow cytometry (FIG. 3E; statistical comparisons are given in FIG. 4C). The average CD8-to-CD4 ratio of T cells co-cultured with Dynabeads under static conditions (n=3) was 2.75±1.5 (mean, ±95% CI). The largest increase in the cellular ratio was observed in the condition where T cells were cultured with 4.5 μm particles with the highest density of ligands, resulting in a CD8-to-CD4 ratio of 30.1±9.8 (p=0.005, compared to static Dynabeads). Averaging across all particle sizes and antigen doses, mechanical oscillation increased the CD8-to-CD4 ratio of the cells 2.1-fold, compared to static culture (ANOVA considering movement, size, and dose; movement p<10⁻¹⁶).

It was noted that the larger particles resulted in more expansion, and especially CD8 expansion, of the T cells than the smaller particles, even though the density of antibodies across the beads of different sizes was almost identical (FIG. 1E) (ANOVA considering size p<2×10⁻¹⁶). This result suggested that the immune synapse integrates the aggregate number of molecular signals across the interface, rather than the density of antigenic ligands.

Methods

Five- to eight-week-old wild-type (C57B1/6) mice were purchased from the Jackson Labs and maintained in specific pathogen-free facilities at UCLA. All experiments on mice and cells collected from mice were performed under an approved protocol of the Animal Research Committee and in accordance with UCLA's institutional policy on humane and ethical treatment of animals. T-cell culture media was RPMI supplemented with 10% heat-inactivated FBS, 1% penicillin/streptomycin, 1% sodium pyruvate, 1% HEPES buffer, 0.1% μM beta-mercaptoethanol. Total T cells, CD4+ T cells or CD8+ T cells were purified using magnetic-based, negative enrichment kits (Stem Cell Technologies). Cells were counted by hemocytometer using trypan blue exclusion (Calbiochem).

Standard Dynabeads (ThermoFisher) or aAPC-assisted in vitro activation of purified T cells was done by culturing cells at a concentration of 1.5×106/mL in 24-well plate. Antibodies employed included anti-CD3 (2C11; Bio X Cell) at a concentration of 10 μg/mL followed by addition of 2 μg/mL soluble anti-CD28 (37.51; Bio X Cell). In indicated conditions, 20 IU/mL of human IL-2 was added. The 4.5 μm microparticle aAPCs or Dynabeads were added to the cells at a 1:1 ratio of particles to cells and other particles (0.8 μm, and 0.3 μm) were added in appropriate concentrations to provide the same surface area.

For flow cytometric analyses, antibodies to mouse CD4, CD8 (53-6.7), CD25 (PC61.5), CD44 (IM7), FoxP3 and CD16/CD32 (“Fc block”) were purchased from eBioscience, BioLegend, or BD Biosciences. To study T-cell proliferative responses in various culture conditions, T-cell expansion was measured by dilution of 5-(and 6-) carboxyfluorescein diacetate, succinimidyl ester (CFSE). For CFSE-dilution experiments, 5×10⁵ cells were labeled with 2 μM CFSE for 13 min at 37° C., washed, and co-cultured with various particles formulations. After the indicated number of days, cells were analyzed by flow cytometry. Flow cytometry was performed on a Cytek DXP 10. FACS data, including calculations regarding proliferation, were analyzed using FlowJo software (Treestar).

### Example 3—Analysis of T Cell Activation with Microparticle Stimulation

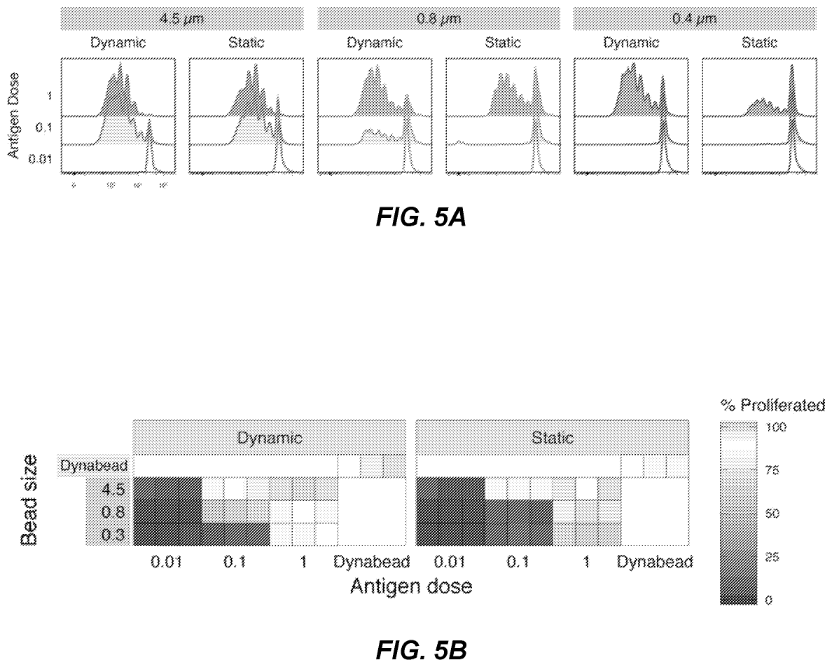

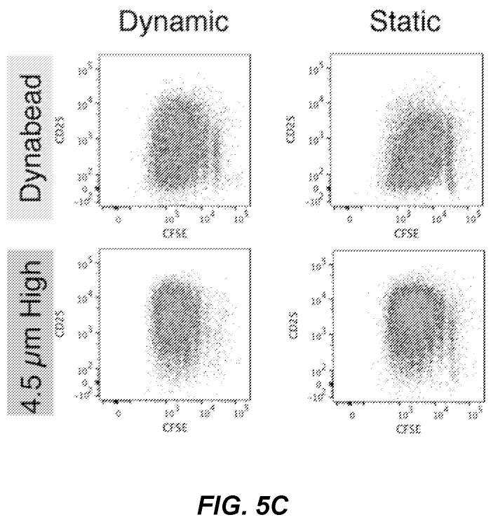

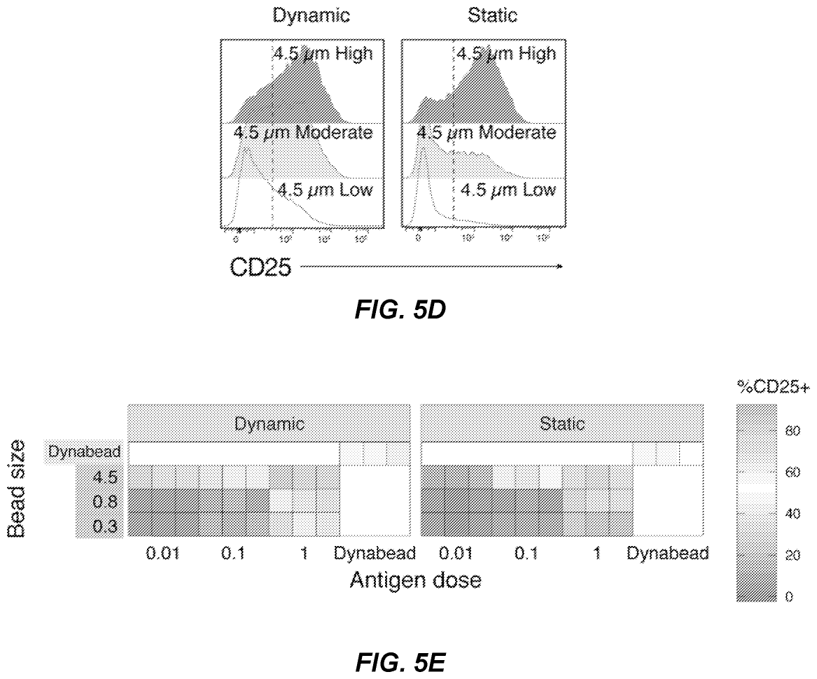

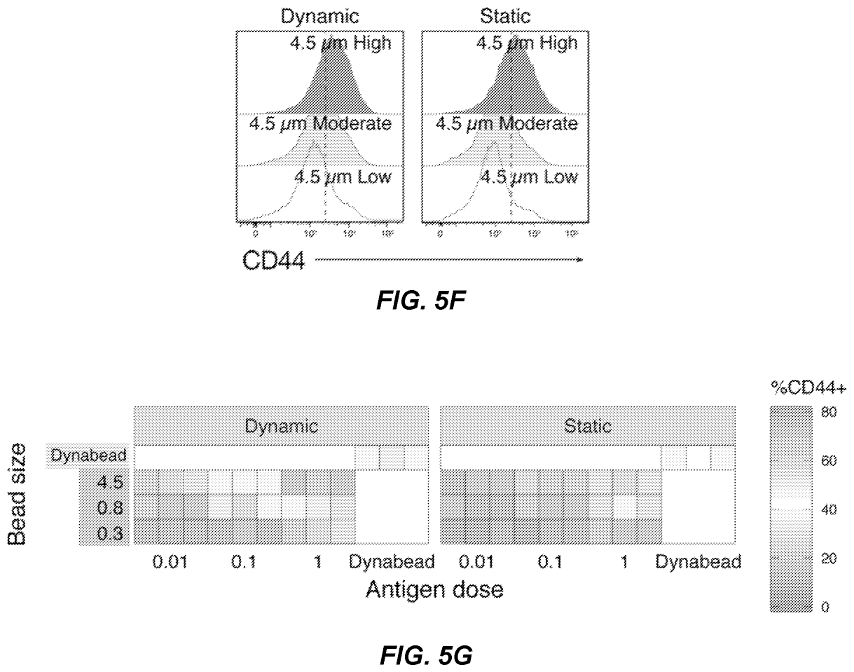

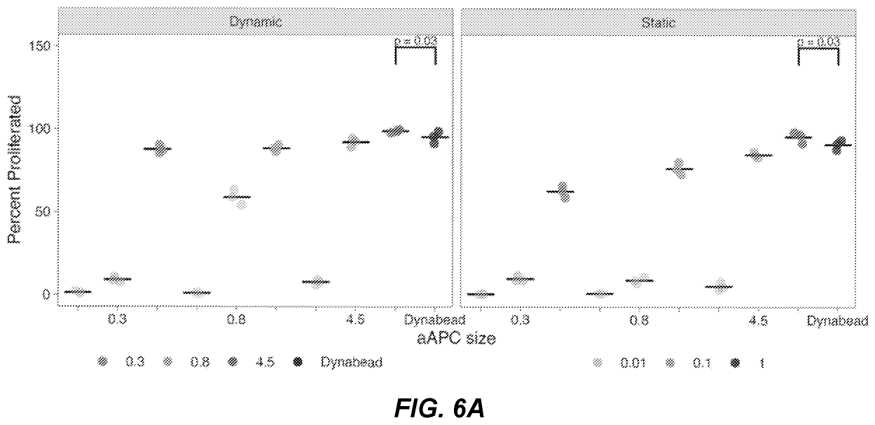

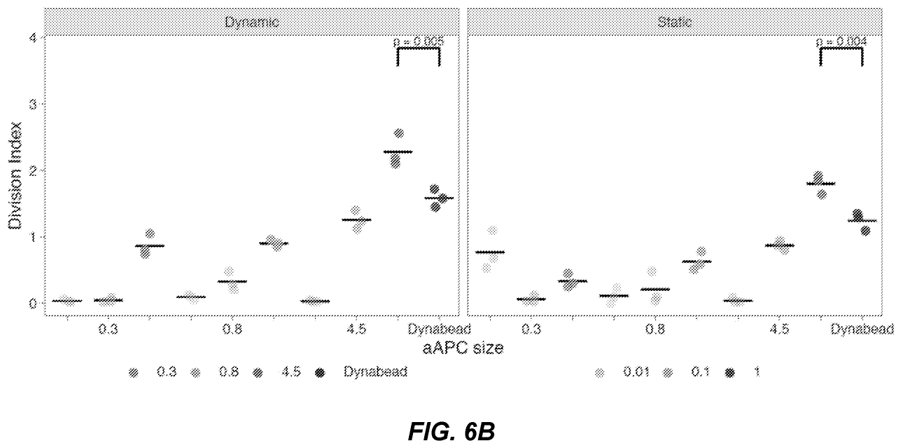

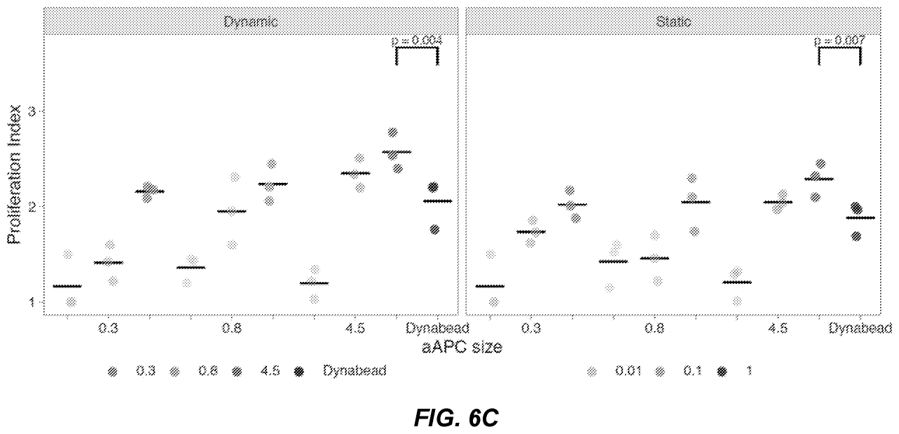

The proliferative responses of T cells upon stimulation with the aAPCs was further examined by using a dye-dilution approach to follow the proliferation pattern (see Example 2 for methods). Sequential generations of daughter cells result in roughly 2-fold dilution of the fluorescent signal (FIG. 5A). The percentage of T cells that underwent proliferation when co-cultured with Dynabeads under static conditions (n=3) was 91.0±5.8% (mean, ±95% CI) (FIG. 5B; statistical comparisons are given in FIG. 6A). The maximum proliferation was observed under the condition where T cells were cultured with 4.5 μm particles with dynamic oscillations at the highest density of antibodies, resulting in proliferation of 98.8±1.9% (p=0.005 compared with static Dynabeads). Averaging across all particle sizes and antigen doses, mechanical oscillation increased the percentage of T cells that underwent proliferation by 1.72-fold, compared to static culture (ANOVA considering movement, size, and dose; movement p=0.011). To measure not just whether the cells divided but also how many times they divided, the division index was also calculated, which is defined as the average number of cell divisions that a T cell in the original population underwent (the average includes cells that never divided at all) (see FIG. 6B in the Supporting Information). Because not all cells proliferated, the proliferation index was also compared, which is defined as the average number of divisions for just the responding population (FIG. 6C in the Supporting Information). These show that the maximum number of divisions was observed under the condition where T cells were dynamically cultured with 4.5 μm particles at the highest density of antibodies.

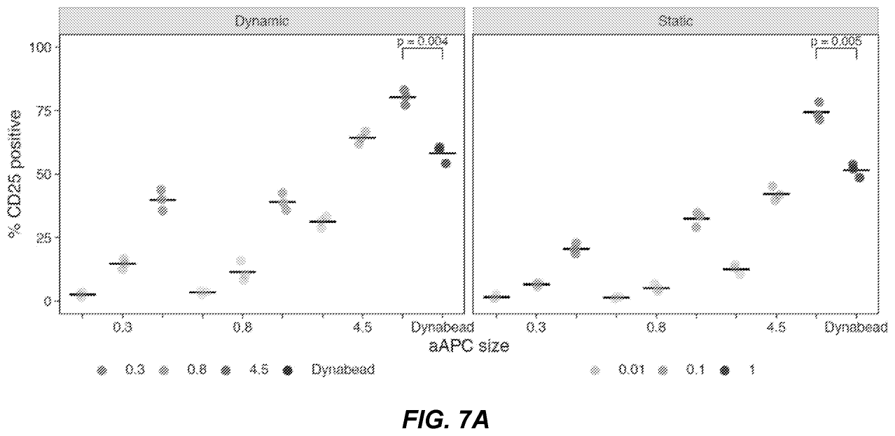

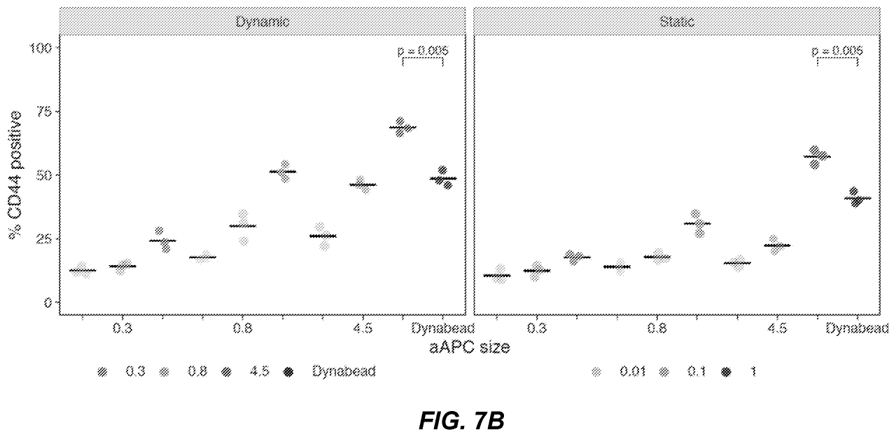

Expression of T-cell activation markers CD25 and CD44 was also examined by flow cytometry after activation and found that expression of these markers trended similarly to proliferation (FIGS. 5D-5G; statistical comparisons are given in FIGS. 7A and 7B). As with absolute expansion, activation and proliferation were greater for larger beads than smaller beads, even when antibody density was held constant. Together, these results showed that activation and proliferation are proportional to the amount of antigen rather than its density.

### Example 4—Evaluation of Synapse Size and Signal Accumulation

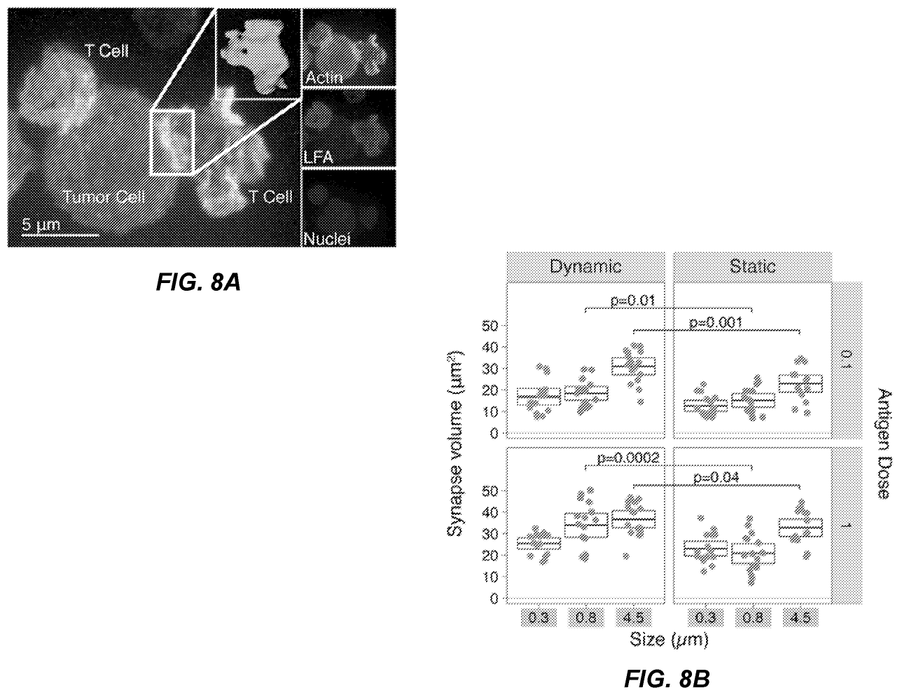

After OT-II T cells were activated with aAPC particles, as in Examples 2 and 3, for 24 h, the stimulatory microparticles were purified away and co-cultured the T cells with cells of the B-cell lymphoma line LB27.4 that were loaded with ovalbumin peptide antigen. The average volume of immune synapses formed between T cells activated under different culture conditions and B cells was measured based on the accumulation of the integrin leukocyte function-associated antigen 1 (LFA-1) measured by the volume of positive pixels (FIG. 8A). The average synapse size for T cells co-cultured with the 4.5 μm microparticles at high levels of stimulatory antibodies under static conditions (n=3) was 32.8±4.1 μm2 (FIG. 8B). The maximum synapse size was observed under the conditions where T cells were cultured with 4.5 μm particles with the high level of antibodies under mechanical oscillation conditions (n=3) 36.7±4.1 μm² (p=0.04, compared to static). Averaging across all particle sizes and antigen doses, mechanical oscillation increased the size of synapses by 1.3-fold, compared to static culture (ANOVA considering movement, size, and dose; movement p=2×10⁻⁷). These results show that the size of the immune synapse is larger when the cells are more activated.

### Example 5—Treg Activation with aAPCs

Results

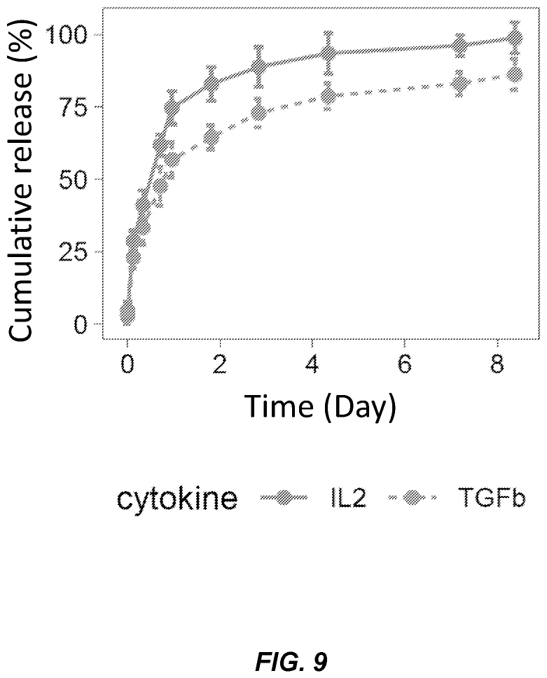

Activating T cells with high signal strength allows for massive expansion, which is needed for transducing chimeric antigen receptors (CARs) and having sufficient transduced cells for a therapeutic dose. The opposite problem arises when expanding T cells in culture for the purposes of generating engineered regulatory T cells. In vivo, regulatory T cells can be elicited to foreign antigens when they are provided at low levels, rather than at high signal strength.⁶ Induction of regulatory T cells is improved by provision of TGF-β and IL-2.18 Alginate microparticles can be loaded with cytokines to skew T cells to induced regulatory T cells (iTregs).⁷ To evaluate the effect of orbital shaking and antigen strength on iTreg formation, Alg-Hep particles were loaded with TGF-β and IL-2. The factors were released from the microparticles over time (shown in FIG. 9). Naive CD4+ T cells were rigorously sorted to eliminate natural regulatory T cells, and then co-cultured these cells with the microparticles. Dynabeads were used for comparison, where an equivalent amount of soluble TGF-β was provided in the media.

The development of Tregs was assessed by intracellular staining for the key transcription factor Foxp3, followed by flow cytometry. The mean fluorescence intensity of the Foxp3 transcript correlates to their regulatory ability,20 and so Foxp3 expression level was measured as well.

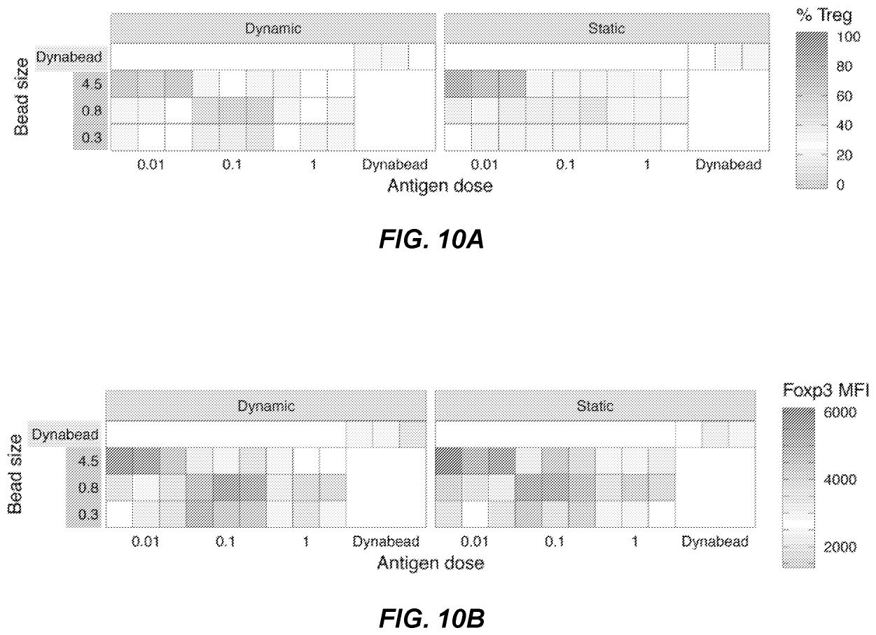

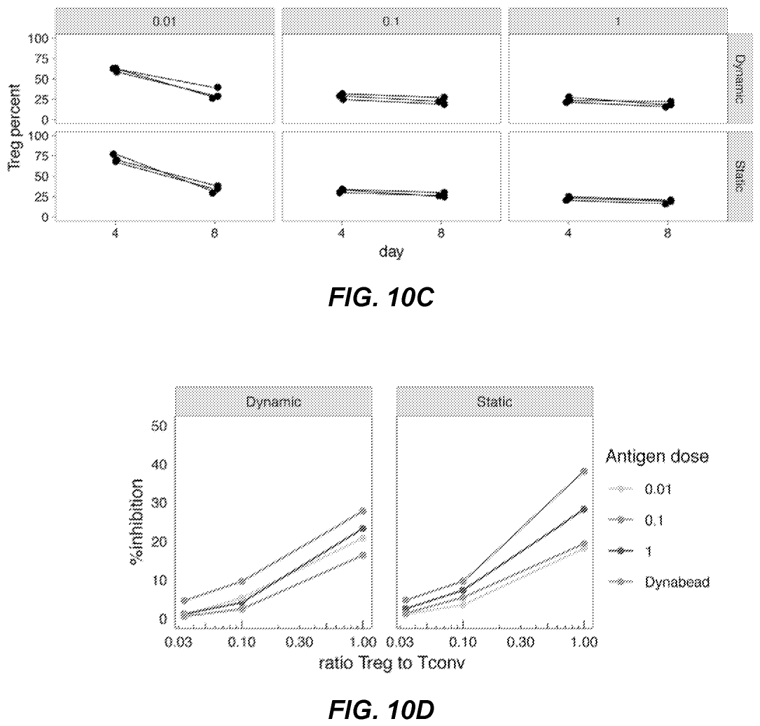

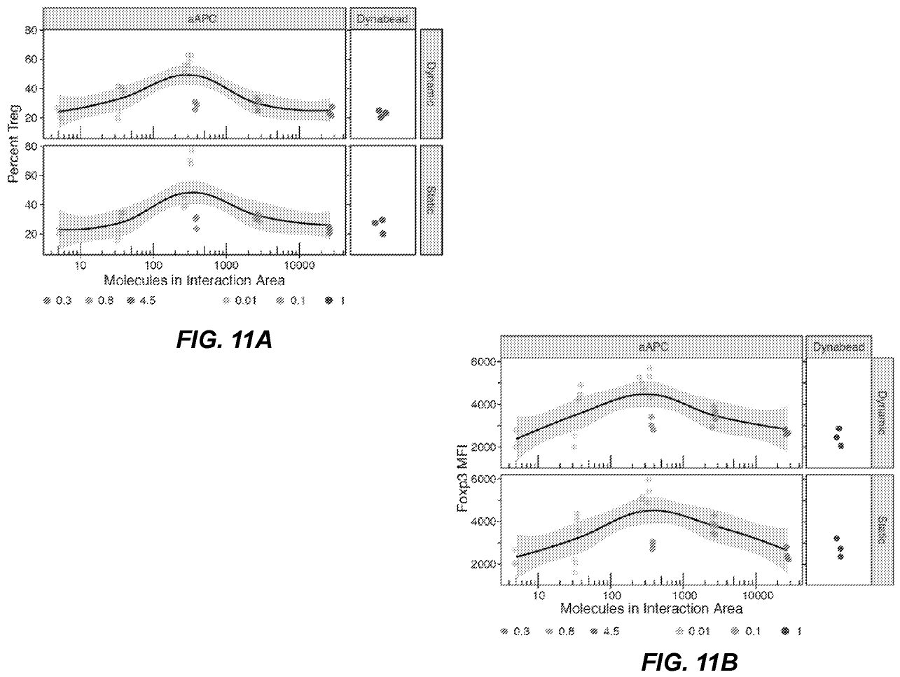

The percentage of iTregs induced in culture with Dynabeads plus TGF-β under static conditions (n=3) was 25.7±10.1% (mean, ±95% CI) (see FIG. 10A). The maximum iTreg induction was observed under the condition where T cells were statically cultured with 4.5 μm microparticles at the lowest density of antibodies (0.01), resulting in iTreg induction of 71.6±9.6%, almost 3-fold higher (p=0.005, compared to static Dynabeads). Generally, averaging across all particle sizes and antigen doses, mechanical oscillation did not alter the rate of iTreg induction (1.02-fold difference). Unexpectedly, the lowest amounts of signal strength, as seen in proliferation and activation assays above, were not able to elicit high yields of iTreg induction. In fact, a “sweet spot” of signal was needed, either in the form of larger beads with lower antibody coating or smaller beads with higher antibody coating amounts (FIG. 11A). The “sweet spot” fell where all three particle sizes could be compared, that is, had comparable numbers of antibodies interacting with T cells (i.e., the largest particles with the lowest antigen density, the medium particles with medium antigen density, and the smallest particles with the highest antigen density). In other words, even as multiple small (300 nm diameter) particles interacted with a single T cell, and offered a comparable antigen amount as a larger particle, the generation of Tregs still favored the situation with the larger particle, i.e., the lower curvature. Thus, in situations where antigen amounts are comparable, larger aAPCs/lower curvature are favored over smaller ones/higher curvature.

The expression of Foxp3 on a per-cell basis was highest under the conditions that elicited the highest induction of iTregs (FIG. 10B), and again showed a “sweet spot” of signal strength (FIG. 11B in the Supporting Information). These results showed that larger immune synapses with low antigen amounts resulted in the highest induction of Tregs.

The stability of the Foxp3 protein expression was examined, since transient expressions of Foxp3 do not yield highly suppressive regulatory T cells.17 The T cells generated through culture with the 4.5 μm microparticles were separated from the microparticles and then were maintained in culture with IL-2. The expression of Foxp3 was assessed by flow cytometry at day 4 and day 8 of culture (FIG. 10C). The expression of Foxp3 was dramatically reduced under the conditions where the induction was highest, that is by the 4.5 μm microparticles that offered the lowest antigen signal (0.01). Dynamic culture mitigated the loss of Foxp3 expression modestly (29.9%±13.1% decrease in dynamic culture versus 37.6%±17.7% in static culture). In contrast, the antigenic strengths that were moderate (0.1) and highest (1) had the most stability in culture (4%-5% diminishment). Overall, at day 8, the highest expression of Foxp3 was still seen in the co-culture conditions with 4.5 μm particles that offered the lowest antigen signal (0.01), 34±8.8% (n=3).

The ultimate in vitro test of regulatory T-cell function is assessed by their ability to suppress the effector responses of conventional, activated T cells. iTregs induced under a variety of conditions were co-cultured with conventional T cells at a cellular ratio of 0, 1, 10, and 30 CFSE-labeled conventional, naive T cells to one iTreg and stimulated with anti-CD3 and anti-CD28. Proliferation of the naive T cells was assessed without Tregs, and the percentage inhibition was measured by subtracting the proliferation as seen when Tregs were co-cultured. Maximal inhibition—and, thus, maximal regulatory function—was enacted by the iTregs that were generated in the “sweet spot” condition of culture with 4.5 μm microparticles at medium levels of activating antibodies (0.1) (FIG. 10D). Together, these results show that the activation and generation of regulatory T cells can be optimized by culturing with particles of large size but low to medium antigenic strength, resulting in the highest stability of regulatory T cells and the most inhibition of effector T-cell proliferation.

Methods

Preparation of Growth Factor Loaded Microparticles

To prepare microparticles loaded with IL-2 and TGF-β, crosslinked particles were incubated with the proteins in PBS buffer containing bovine serum albumin (BSA; 0.1% w/v) and were gently shaken overnight at 4° C. The particles were then centrifuged and washed several times to remove unabsorbed proteins. The concentrations of IL-2 and TGF-β in the removed supernatant were measured using enzyme-linked immunosorbent assay (ELISA) to estimate the binding capacity of particles. To study the in vitro release profile, protein-loaded particles were dispersed in PBS (pH 7.4) and 500 μL of particles were placed in Eppendorf tubes, gently shaken, and incubated at 37° C. At predetermined time points, samples were collected using centrifugation and the supernatant was replaced with an equivalent volume of fresh PBS solution.

Treg Isolation and Activation

For experiments to induce regulatory T cells (iTreg), CD4+ T cells were purified from mouse spleens by EasySep immunomagnetic negative selection (Stem Cell Technologies). Cells were then activated on anti-CD3e antibody (10 μm/mL)-coated plates with media supplemented with anti-CD28 antibody (2 μg/mL). At the same time particles loaded with TGF-β and IL-2 were added to the media. After four days regulatory T cells were removed from wells coated with anti-CD3e and fixed, permeabilized, and stained with antibodies for flow cytometry analysis. Stability of formed iTregs were also tested after 4 and 8 days of culture by flow cytometry in a similar fashion.

In Vitro Treg Suppression Assay

iTregs were generated using the above approach and flow sorted by expression of CD4+CD25^{hi} cells. To prepare naïve, conventional T cells (Tconv), CD4+ T cells from mouse spleens underwent flow sorting to eliminate all CD25-positive cells, which eliminated activated and natural Tregs (nTregs). iTreg and CFSE-labeled naïve Tconv were then combined at three different ratios of iTreg:Tconv (1:1, 1:10, and 1:30) while being stimulated with plate-coated anti-CD3 and soluble anti-CD28 in 24-well plates as above. As a control, T-cell cultures without iTregs were stimulated in the same manner, and % inhibition was calculated by comparing these cultures to those containing iTregs. Activation markers for iTregs and CD4+ T cells were tested as mentioned before. Flow sorting was performed on a Sony SH800 sorter.

### Example 6—Evaluation of Oscillatory Stimulation

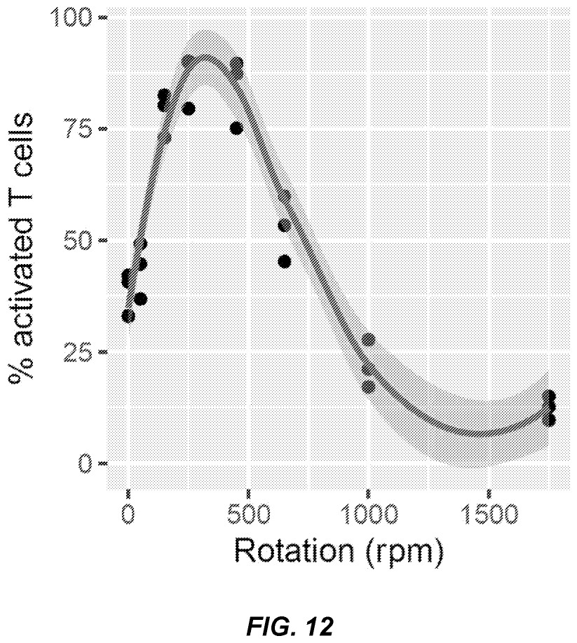

T cells were incubated with antibody-coated microparticles of the present disclosure, as described in Example 2, at speeds of between 0 and 1750 rotations per minute (rpm). The results are shown in FIG. 12. The highest % T cell activation was observed at speeds of between about 200 rpm and about 500 rpm.

### * * *

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Xu, J.; Melenhorst, J. J.; Fraietta, J. A. Toward Precision Manufacturing of Immunogene T-Cell Therapies. Cytotherapy 2018, 20, 623-638.
2. Reth, M. Matching Cellular Dimensions with Molecular Sizes. Nat. Immunol. 2013, 14, 765-767.
3. Thauland, T. J.; Hu, K. H.; Bruce, M. A.; Butte, M. J. Cytoskeletal Adaptivity Regulates T Cell Receptor Signaling. Sci. Signaling 2017, 10, eaah3737.
4. Irvine, D. J.; Purbhoo, M. A.; Krogsgaard, M.; Davis, M. M. Direct Observation of Ligand Recognition by T Cells. Nature 2002, 419, 845-849.
5. Manz, B. N.; Jackson, B. L.; Petit, R. S.; Dustin, M. L.; Groves, J. T-Cell Triggering Thresholds Are Modulated by the Number of Antigen within Individual T-Cell Receptor Clusters. Proc. Natl. Acad. Sci. U.S.A. 2011, 108, 9089-9094.
6. Rubtsov, Y. P.; Niec, R. E.; Josefowicz, S.; Li, L.; Darce, J.; Mathis, D.; Benoist, C.; Rudensky, A. Y. Stability of the Regulatory T Cell Lineage in Vivo. Science 2010, 329, 1667-1671.
7. Majedi, F. S.; Hasani-Sadrabadi, M. M.; Kidani, Y.; Thauland, T. J.; Moshaverinia, A.; Butte, M. J.; Bensinger, S. J.; Bouchard, L.-S. Cytokine Secreting Microparticles Engineer the Fate and the Effector Functions of T-Cells. Adv. Mater. 2018, 30, 1703178.
8. Perica, K.; Kosmides, A. K.; Schneck, J. P. Linking Form to Function: Biophysical Aspects of Artificial Antigen Presenting Cell Design. Biochim. Biophys. Acta, Mol. Cell Res. 2015, 1853, 781-790.

What is claimed is:

1. A method for activating an immune cell comprising:
(a) generating a mixture comprising (i) the immune cell and (ii) an antibody-coated microparticle; and
(b) providing an external mechanical stimulation to the mixture.

2. The method of claim 1, further comprising, prior to (a), obtaining the immune cell from a subject.

3. The method of claim 2, wherein obtaining the immune cell comprises isolating the immune cell from a biological sample from the subject.

4. The method of claim 3, wherein the biological sample is a blood sample or a plasma sample.

5. The method of claim 4, wherein the immune cell is isolated from peripheral blood mononuclear cells from the subject.

6. The method of claim 3, wherein the biological sample is a biopsy sample.

7. The method of any of claims 1-6, further comprising, following (b), providing the immune cell to a subject.

8. The method of claim 7, further comprising providing to the subject an additional therapy.

9. The method of claim 8, wherein the additional therapy is an immunotherapy.

10. The method of any of claims 7-9, wherein the immune cell was obtained from the subject.

11. The method of any of claims 7-9, wherein the immune cell was not obtained from the subject.

12. The method of any of claims 2-11, wherein the subject suffers from or is suspected of having cancer.

13. The method of any of claims 2-12, wherein the subject suffers from or is suspected of having a viral infection.

14. The method of any of claims 1-13, wherein providing the external mechanical stimulation to the mixture generates a population of activated immune cells from the immune cell.

15. The method of claim 14, further comprising isolating the activated immune cells from the mixture.

16. The method of claim 14 or 15, further comprising providing the activated immune cells to the subject.

17. The method of any of claims 1-16, wherein the immune cell is a T cell.

18. The method of claim 17, wherein the T cell is a cytotoxic T cell.

19. The method of claim 17, wherein the T cell is a CD4+ T cell.

20. The method of claim 17, wherein the T cell is a CD8+ T cell.

21. The method of any of claims 1-20, wherein the mechanical stimulation is an oscillatory stimulation.

22. The method of claim 21, wherein the oscillatory stimulation is provided at between 150 rotations per minute (rpm) and 500 rpm.

23. The method of claim 22, wherein the oscillatory stimulation is provided at about 250 rpm.

24. The method of any of claims 1-23, wherein the microparticle comprises antibodies.

25. The method of claim 24, wherein the microparticle comprises at least 200 fg of antibodies.

26. The method of claim 25, wherein the microparticle comprises at least 500 fg of antibodies.

27. The method of claim 26, wherein the microparticle comprises at least 750 fg of antibodies.

28. The method of any of claims 1-27, wherein the microparticle comprises anti-CD3 antibodies, anti-CD28 antibodies, anti-CD137 antibodies, or a combination thereof.

29. The method of claim 28, wherein the microparticle comprises anti-CD3 and anti-CD28 antibodies.

30. The method of any of claims 1-29, wherein the microparticle has a stiffness of between 10 kPa and 30 kPa.

31. The method of claim 30, wherein the microparticle has a stiffness of about 20 kPa.

32. The method of any of claims 1-31, wherein the microparticle is between 0.2 μm and 5.0 μm in diameter.

33. The method of any of claims 1-32, wherein the microparticle is an alginate microparticle.

34. The method of any of claims 1-33, wherein the microparticle comprises a magnetic nanoparticle.

35. The method of claim 34, where the magnetic nanoparticle is encapsulated within the microparticle.

36. The method of claim 34 or 35, wherein the microparticle comprises a plurality of magnetic nanoparticles.

37. The method of any of claims 1-36, wherein the external mechanical stimulation is provided for at least 12 hours.

38. The method of claim 37, wherein the external mechanical stimulation is provided for at least 24 hours.

39. The method of claim 38, wherein the external mechanical stimulation is provided for at least 72 hours.

40. The method of any of claims 1-39, wherein, during (b), the immune cell expands at least 10-fold.

41. A method of treating a subject for cancer, the method comprising:
(a) generating a mixture comprising (i) an immune cell and (ii) an antibody-coated microparticle;
(b) providing an external mechanical stimulation to the mixture to generate activated immune cells from the immune cell; and
(c) providing the activated immune cells to the subject.

42. The method of claim 41, wherein the activated immune cells are activated T cells.

43. The method of claim 41 or 42, further comprising, prior to (c), inserting a nucleic acid encoding for a therapeutic protein into the activated immune cells to generate therapeutic immune cells expressing the therapeutic protein.

44. The method of any of claims 41-43, wherein the therapeutic protein is a chimeric antigen receptor.

45. A method for activating a regulatory T cell (Treg), the method comprising generating a mixture comprising (i) the Treg and (ii) an antibody-coated microparticle comprising between 0.5 and 100 fg of antibodies.

46. The method of claim 45, further comprising, prior to (a), obtaining the Treg from a subject.

47. The method of claim 45, further comprising, following (b), providing the Treg to a subject.

48. The method of claim 47, wherein the Treg was obtained from the subject.

49. The method of claim 47, wherein the Treg was not obtained from the subject.

50. The method of any of claims 45-49, wherein the subject suffers from or is suspected of having an autoimmune disorder.

51. The method of any of claims 45-50, wherein the Treg is an induced Treg.

52. The method of any of claims 45-51, wherein activated Tregs are generated from the Treg.

53. The method of claim 52, further comprising isolating the activated Tregs from the mixture.

54. The method of claim 53, further comprising providing the activated Tregs to the subject.

55. The method of any of claims 45-54, further comprising providing an external mechanical stimulation to the mixture.

56. The method of claim 55, wherein the mechanical stimulation is an oscillatory stimulation.

57. The method of claim 56, wherein the oscillatory stimulation is provided at between 150 rotations per minute (rpm) and 500 rpm.

58. The method of claim 57, wherein the oscillatory stimulation is provided at about 250 rpm.

59. The method of any of claims 45-58, wherein the microparticle comprises antibodies.

60. The method of claim 59, wherein the microparticle comprises between 1 and 50 fg of antibodies.

61. The method of claim 60, wherein the microparticle comprises between 15 and 40 fg of antibodies.

62. The method of claim 61, wherein the microparticle comprises between 20 and 30 fg of antibodies.

63. The method of any of claims 59-62, wherein the microparticle comprises anti-CD3 antibodies, anti-CD28 antibodies, anti-CD137 antibodies, or a combination thereof.

64. The method of claim 63, wherein the microparticle comprises anti-CD3 and anti-CD28 antibodies.

65. The method of any of claims 45-64, wherein the microparticle has a stiffness of between 10 kPa and 30 kPa.

66. The method of claim 65, wherein the microparticle has a stiffness of about 20 kPa.

67. The method of any of claims 45-66, wherein the microparticle is between 0.2 μm and 5.0 μm in diameter.

68. The method of any of claims 45-67, wherein the microparticle is greater than 4 μm in diameter and has an antibody density of less than 200 proteins per μm² on its surface.

69. The method of any of claims 45-67, wherein the microparticle is less than 1 μm and has an antibody density of between 100 and 500 proteins per μm² on its surface.

70. The method of any of claims 45-69, wherein the microparticle is an alginate microparticle.

71. The method of any of claims 45-70, wherein the microparticle comprises a magnetic nanoparticle.

72. The method of claim 71, where the magnetic nanoparticle is encapsulated within the microparticle.

73. The method of claim 71 or 72, wherein the microparticle comprises a plurality of magnetic nanoparticles.

74. The method of any of claims 45-73, wherein the microparticle comprises one or more growth factors capable of stimulating a signaling pathway in the Treg.

75. The method of claim 74, wherein the one or more growth factors are encapsulated within the microparticle.

76. The method of claim 74 or 75, wherein the one or more growth factors comprise TGF-β or IL2.

77. The method of any of claims 45-76, wherein the external mechanical stimulation is provided for at least 12 hours.

78. The method of claim 77, wherein the external mechanical stimulation is provided for at least 24 hours.

79. The method of claim 78, wherein the external mechanical stimulation is provided for at least 72 hours.

80. The method of any of claims 45-79, wherein, during (b), the Treg expands at least 10-fold.

81. A method for treating a subject for an autoimmune disorder, the method comprising:
(a) generating a mixture comprising (i) a regulatory T cell (Treg) and (ii) an antibody-coated microparticle comprising between 0.5 and 100 fg of antibodies;
(b) generating activated Tregs from the Treg; and
(c) providing the activated Tregs to the subject.

82. The method of claim 81, wherein the Treg is an induced Treg.

83. The method of claim 81 or 82, further comprising isolating the activated Tregs from the mixture.

84. A method for activating a population of T cells, the method comprising:
(a) generating a mixture comprising (i) the T cells and (ii) antibody-coated microparticles comprising, on average, at least 200 fg of antibodies per microparticle;
(b) providing an external oscillatory stimulation to the mixture at a speed of between 100 and 500 rotations per minute, thereby generating a population of activated T cells; and
(c) isolating the activated T cells from the mixture.

85. A method for activating a population of regulatory T cell (Tregs), the method comprising generating a mixture comprising:
(a) the Tregs; and
(b) antibody-coated microparticles, wherein the antibody-coated microparticles are, on average:
(i) greater than 4 μm in diameter and have an antibody density of less than 200 proteins per μm² on their surface; or
(ii) less than 1 μm and have an antibody density of between 100 and 500 proteins per μm² on their surface.
